(19) United States
(12) Patent Application Publication
(10) Pub. No.: US 2026/0123930 A1
(43) Pub. Date: May 7, 2026

(54) MONITORING OF AN ACTUATION STROKE WITH A RELOCATABLE MAGNET IN A SURGICAL INSTRUMENT HANDLE

(71) Applicant: Cilag GmbH International, Zug (CH)

(72) Inventors: Matthew D. Cowperthwait, Cincinnati, OH (US); Frederick E. Shelton, IV, Hillsboro, OH (US); Thomas A. Stoughton, Cincinnati, OH (US); Seth D. Holdmeyer, Cincinnati, OH (US); Shane R. Adams, Lebanon, OH (US); Karl W. Mueller, Cincinnati, OH (US); Nicholas J. Ross, Franklin, OH (US)

(21) Appl. No.: 18/939,013

(22) Filed: Nov. 6, 2024

(51) Int. Cl. A61B 17/072; A61B 17/00; A61B 17/128

(52) U.S. Cl. CPC A61B 17/07207; A61B 17/128; A61B 2017/00022; A61B 2017/0046

(57) ABSTRACT

A surgical instrument comprising a handle and a reusable housing. The handle comprises a frame supporting a trigger and a magnet. Actuation of the trigger relocates the magnet relative to the frame. The reusable housing supports a circuit and a sensor in signal communication with the circuit. The reusable housing is detachably mounted to the handle and the sensor is configured to detect a signal based on movement of the magnet. The circuit is configured to determine directionality and degree of movement of the trigger based on the signal detected by the sensor.

### BACKGROUND

Disposable electronic surgical devices offer convenience but generate significant waste. To address this issue, it is desirable to separate the device into disposable components and reusable components. To be viable, the reusable components should be reusable after completion of a sterilization process, for example. For optimal cost efficiency, the electronic components, which are costly to replace, should be reusable. To achieve this, the electronic components should be sealed within a housing that withstands sterilization processes, such as the high temperatures and pressures associates with autoclaving, for example. This approach can reduce waste and enhance cost-effectiveness.

A challenge arises because electronic components are often characterized or otherwise tuned to different specifications for each electronic surgical device. For example, U.S. Pat. No. 9,808,244, titled SENSOR ARRANGEMENTS FOR ABSOLUTE POSITIONING SYSTEM FOR SURGICAL INSTRUMENTS, issued Nov. 7, 2017, discloses absolute positioning systems to provide a unique position signal over one revolution of rotary motion. U.S. Pat. No. 9,808,244, titled SENSOR ARRANGEMENTS FOR ABSOLUTE POSITIONING SYSTEM FOR SURGICAL INSTRUMENTS, issued Nov. 7, 2017 is incorporated by reference herein in its entirety.

To reuse electronic components, the surgical device should detect threshold parameters and/or discrete states without requiring the electronic component to be calibrated to each disposable component to which it is coupled.

### SUMMARY

In one aspect, the present disclosure provides a surgical instrument comprising a handle comprising a frame supporting a trigger and a magnet, wherein an actuation of the trigger relocates the magnet relative to the frame. The surgical instrument comprises a reusable housing supporting a circuit and a sensor in signal communication with the circuit. The reusable housing is detachably mounted to the handle and the sensor is to detect a signal based on relocation of the magnet. The circuit is to determine directionality and degree of movement of the trigger based on the signal detected by the sensor.

In another aspect, the present disclosure provides a surgical instrument comprising a handle comprising a frame supporting a trigger and a magnet. An actuation of the trigger relocates the magnet relative to the frame. The surgical instrument comprises a reusable housing detachably mounted to the handle, the reusable housing supporting a circuit, a first sensor to detect a first signal based on a positional movement of the magnet, and a second sensor to detect a second signal based on a directional movement of the trigger. The first sensor and the second sensor are in signal communication with the circuit. The circuit is to determine a closure state of the surgical instrument based on the first signal and the second signal.

In another aspect, the present disclosure provides a surgical instrument comprising a handle comprising a frame supporting a trigger and a magnet. An actuation of the trigger relocates the magnet relative to the frame. The surgical instrument comprises a reusable housing supporting a circuit. The reusable housing is detachably mounted to the handle. The surgical instrument comprises a sensor in signal communication with the circuit. The sensor is to detect a signal based on movement of the magnet. The circuit is to determine directionality and degree of movement of the trigger based on the signal detected by the sensor.

### BRIEF DESCRIPTION OF THE DRAWINGS

In the description, for purposes of explanation and not limitation, specific details are set forth, such as particular aspects, procedures, techniques, etc. to provide a thorough understanding of the present technology. However, it will be apparent to one skilled in the art that the present technology may be practiced in other aspects that depart from these specific details.

The accompanying drawings, where like reference numerals refer to identical or functionally-similar elements throughout the separate views, together with the detailed description below, are incorporated in and form part of the specification, and serve to further illustrate aspects of concepts that include the claimed disclosure and explain various principles and advantages of those aspects.

The surgical devices disclosed herein have been represented where appropriate by conventional symbols in the drawings, showing only those specific details that are pertinent to understanding the various aspects of the present disclosure so as not to obscure the disclosure with details that will be readily apparent to those of ordinary skill in the art having the benefit of the description herein.

FIG. 1 is a perspective view of a surgical instrument including a reusable housing separated from a replaceable handle, in accordance with at least one aspect of the present disclosure.

FIG. 2 is a perspective view of the surgical instrument of FIG. 1 with a portion of the casing of the replaceable handle removed for illustrative purposes, in accordance with at least one aspect of the present disclosure.

FIG. 3 is a perspective view of a portion of the surgical instrument of FIG. 1 with a portion of the casing of the replaceable handle removed for illustrative purposes and depicting a link connected to a clamp arm, and further depicting a trigger release actuator comprising a cam follower arm slidably engaged with the link, in accordance with at least one aspect of the present disclosure.

FIG. 4 is a side profile view of the link of FIG. 3 and a portion of the cam follower arm of FIG. 3, and further depicting an alternative geometric profile of the link with dashed lines, in accordance with at least one aspect of the present disclosure.

FIG. 5 is a graphical representation of signals detected by a sensor as the clamp arm of FIG. 3 moves through a closure stroke, in accordance with at least one aspect of the present disclosure.

FIG. 6 is a side view of a surgical instrument comprising a clamp arm and a cam movable with the clamp arm, the surgical instrument further comprising an arm supporting a sensor and the surgical instrument being depicted in a first configuration, in accordance with at least one aspect of the present disclosure.

FIG. 7 is a side view of the surgical instrument of FIG. 6, depicting the surgical instrument in a second configuration, in accordance with at least one aspect of the present disclosure.

FIG. 8 is a graphical representation of a signal detected by the sensor of FIG. 6 as the clamp arm of the surgical instrument of FIG. 6 is moved between the first configuration depicted in FIG. 6 and the second configuration depicted in FIG. 7, in accordance with at least one aspect of the present disclosure.

FIG. 9 is a detent and a pawl positioned to create a voltage spike detectable by a sensor during an operational stroke of a surgical instrument, in accordance with at least one aspect of the present disclosure.

FIG. 10 is a graphical representation of a signal detect by the sensor of FIG. 9, in accordance with at least one aspect of the present disclosure.

### DETAILED DESCRIPTION

Reusable electronics for a surgical instrument or surgical system can include a printed circuit board assembly (PCBA) and supercapacitors, for example. Such reusable electronics can be referred to as an electronics package. The electronics package can be used to power and control multiple surgical devices, which can be replaceable and/or disposable after each use (e.g. single-use surgical devices). The replaceable components can have different variances, which may cause inaccurate signal readings for a reusable control circuit that has been pre-calibrated or characterized to another previously-used replaceable component. In turn, the reusable control circuit may improperly interpret a signal as being indicative of a property or state of the surgical device that is not accurate of the surgical device's actual property or state. For example, a signal from a first replaceable, articulatable surgical stapling instrument may be indicative of a different articulation position than the same signal when connected to a second replaceable, articulatable surgical stapling instrument due to tolerances in the manufacture and assembly of the surgical instruments and, for example, the articulation systems thereof. Determining how to characterize the various electronically-monitored features of a surgical device relative to the reusable electronics package may be important in ensuring that the same electronics package can be reused with multiple devices without characterizing or calibrating the electronics package to each unique device.

A surgical device can utilize electronically-monitored features to determine the operational state of the surgical device and to selectively lock, unlock, activate, and/or deactivate one or more surgical functions according to a predefined sequence of surgical events or functions. For example, a staple firing stroke can be prevented until the jaws of the stapling end effector are fully clamped onto tissue. As another example, an articulation motion of an articulatable end effector can be prevented when the end effector is partially- or fully-clamped onto tissue. Monitored operational states and threshold parameters of the surgical device can include, for example, the position of a firing bar or firing rack, the articulation angle of an end effector, and/or the rotational position of a manually-operated actuator, such as a closure trigger and/or firing trigger, for example.

Surgical devices are disclosed that indicate operational states and threshold conditions based on sensed parameters, which can eliminate the need for characterization of the reusable electronics relative to the replaceable, disposable portion of the surgical device. In various aspects, the surgical devices include a magnetic sensor that determines the directionality and degree of movement of an electronically-monitored structure of the replaceable, disposable portion of the surgical device, such as a manual actuator or driver of a surgical stapler, for example. In various aspects, the disposable portion of the surgical device may include a magnet that is operably coupled to an actuator of the disposable portion of the surgical device such that the magnet is relocated upon actuation of the actuator. Upon actuation of the actuator, and, in turn, movement of the magnet, a sensor within the reusable electronics package may detect a signal, such as a voltage change, as the actuator moves through the actuation stroke or a portion of the actuation stroke. The signal detected by the sensor may be indicative of a particular direction and degree of movement of the actuator. Based on the direction and degree of movement of the actuator, a control circuit within the reusable electronics package can determine an operational state of the end effector of the surgical device. Depending on the state of the end effector, the circuit may engage or disengage certain functions of the surgical device, such as allowing activation of the firing system and/or articulation of the end effector, for example.

Magnetic sensing systems are effective in surgical devices including a reusable electronics package because magnetic sensing systems are able to transmit an electromagnetic field across a distance and between separable components to produce a signal. Wired connections are generally impractical in such environments because the reusable housing supporting the electronic package should be sealed to withstand sterilization processes, such as autoclaving, for example. As such, in surgical devices where the electronics package is separable from the surgical device for sterilization and reuse, for example, the control circuit should be able to receive inputs from the user-operated surgical device across the seal to control the motors and other electronic functions. This can be achieved via magnetic sensing, for example.

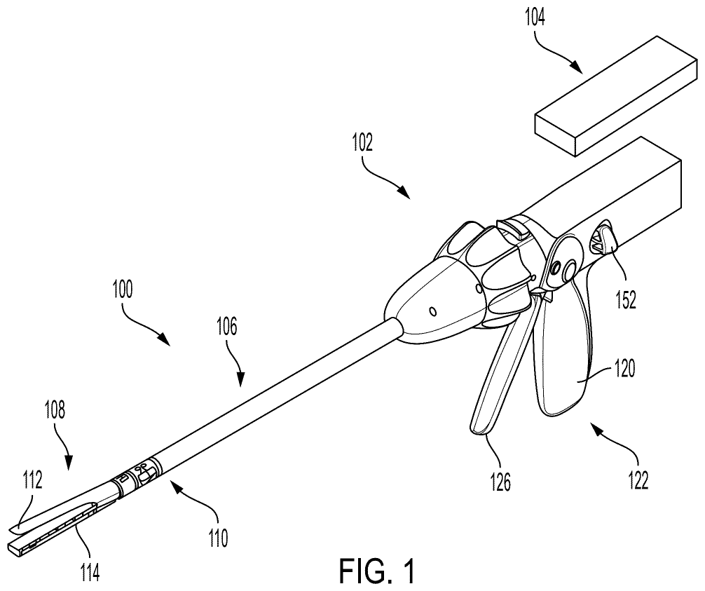

Referring primarily to FIGS. 1-4, a surgical instrument 100 includes disposable components and reusable components, which can enhance convenience and/or reduce waste. The reusable components are designed to endure sterilization processes, such as autoclaving, for example, for reuse during a subsequent surgical procedure. The surgical instrument includes a replaceable handle 102 and a reusable housing 104 detachably mounted to the replaceable handle 102. The replaceable handle 102 can be a single-use, disposable component, whereas the reusable housing 104 can be a reusable component that is used sequentially with multiple replaceable handles, for example. The replaceable handle 102 can be releasably secured to the reusable housing 104 with a latch or clasp, for example.

The surgical instrument 100 includes a shaft 106 and an end effector 108 pivotally connected to the shaft 106 at an articulation joint 110. The end effector 108 includes a first jaw 112 and a second jaw 114. At least one of the first jaw 112 and the second jaw 114 is movable relative to the other to clamp tissue therebetween. The end effector 108 is designed to clamp, sever, and staple tissue. In such instances, the end effector 108 can be referred to as a stapling end effector and the surgical instrument 100 can be referred to as surgical stapler. In other aspects of the present disclosure, the end effector 108 can be configured to perform additional and/or alternative surgical functions such as grasping, cutting, clamping, guiding, imaging, providing tool access, delivering drug and/or gene therapy, and/or energizing with ultrasonic, radio frequency (RF), and/or laser energy, for example.

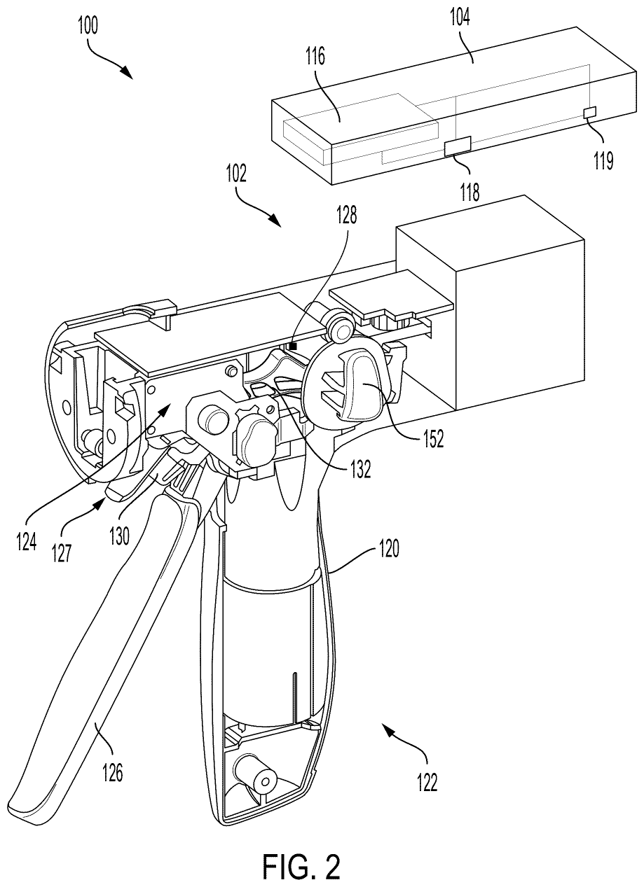

As shown in FIG. 2, the reusable housing 104 supports an electronics package that includes a circuit board 116 and a sensor 118 in signal communication with the circuit board 116. The circuit board 116 can be a PCBA, for example. The circuit board supports a circuit, which can also be referred to a controller circuit, which can include a microprocessor and a memory. In various embodiments, the reusable housing 104 can support multiple sensors, such as a first sensor 118 and a second sensor 119 in signal communication with the processor and, in various instances, mounted to the circuit board 116. Because the reusable housing 104 is detachably mounted to the replaceable handle 102, the reusable housing 104 is sealed to withstand sterilization processes, such as autoclaving, for example.

The replaceable handle 102 includes two housing segments 120 that interconnect and cooperate to form a pistol grip 122. The pistol grip 122 is shaped and dimensioned to be gripped and manipulated by a clinician. The housing segments 120 can be connected by adhesive and/or mechanical fasteners, such as screws, snap features, etc. The housing segments 120 operably support one or more drive systems in the pistol grip 122. For example, a closure drive system, a firing drive system, and an articulation systems are supported in the interconnected housing segments. The drive systems are configured to generate, transmit, and apply various control motions along the shaft 106 to the end effector 108. Exemplary drive systems are further described in, for example, U.S. Pat. No. 9,808,244, titled SENSOR ARRANGEMENTS FOR ABSOLUTE POSITIONING SYSTEM FOR SURGICAL INSTRUMENTS, which issued Nov. 7, 2017.

As shown in FIG. 2, the replaceable handle 102 of the surgical instrument 100 includes a frame 124 to operably support at least a portion of the drive system(s) and the components thereof. For example, the frame 124 supports a closure drive system, which is actuatable to apply closing and opening motions to the end effector 108. In at least one form, the closure drive system may include a manual actuator or trigger in the form of a clamp arm, or clamp lever, 126, which is pivotally supported by the frame 124. More specifically, the clamp arm 126 is pivotally supported by the frame 124 such that when the clinician grips the pistol grip 122 of the replaceable handle 102, the clamp arm 126 is accessible to the clinician and easily pivotable relative to the frame 124 and the pistol grip 122 from an unactuated position to an actuated position. The clamp arm 126 is biased into the unactuated position. For example, a spring or other biasing arrangement can bias the clamp arm 126 toward the unactuated position and a clinician can overcome the biasing force to move the clamp arm 126 toward the actuated position.

The frame 124 also supports a firing drive system, which is configured to apply firing motions to end effector 108. In at least one form, the firing drive system may include an actuator in the form of a firing trigger 127 that is pivotally supported by the frame 124. The firing trigger 127 may also be pivoted between an unactuated position and an actuated position. A spring or other biasing arrangement can bias the firing trigger 127 into the unactuated position such that, upon release of the firing trigger 127 by the clinician, the firing trigger 127 is pivoted or otherwise returned to the unactuated position.

A safety actuator 130 is also pivotally connected to the frame 124 and is positioned between the clamp arm 126 and the firing trigger 127. When the clamp arm 126 is in the unactuated position, the safety actuator 130 is contained, or substantially contained, within the housing segments 120 and is inaccessible or not readily accessible to the clinician. As the clinician depresses the clamp arm 126, the safety actuator 130 and the firing trigger 127 can pivot outward such that the trigger 127 and the safety actuator 130 are positioned to be manipulated by the clinician. The safety actuator 130, in the unactuated position, is structured and positioned to mechanically prevent actuation of the firing trigger 127.

The frame 124 supports the clamp arm 126 for pivotal motion relative to the frame 124 upon actuation by a clinician to operate a drive system. The frame 124 also supports a magnet 128, such that actuation of the clamp arm 126 relocates the magnet 128 relative to the frame 124 during at least a portion of the closure stroke. The magnet 128 is a permanent magnet. The magnet 128 can be relocated relative to the frame 124 by displacing the magnet 128 as a whole to a new location relative to the frame 124. For example, the magnet 128 can be fixed to a moving (e.g. translating and/or pivoting) component in the replaceable handle 102 to relocate the entire magnet 128 along a linear and/or contoured path as the component moves. The path along which the magnet 128 is relocated can extend in two-dimensions or three-dimensions. Relocation of the magnet 128 along a two-dimensional path or a three-dimensional-path involves more than rotation of the magnet (and positive and negative poles thereof) about a fixed point. In other words, the entire magnet is moved relative to the frame 124, not merely the poles thereof.

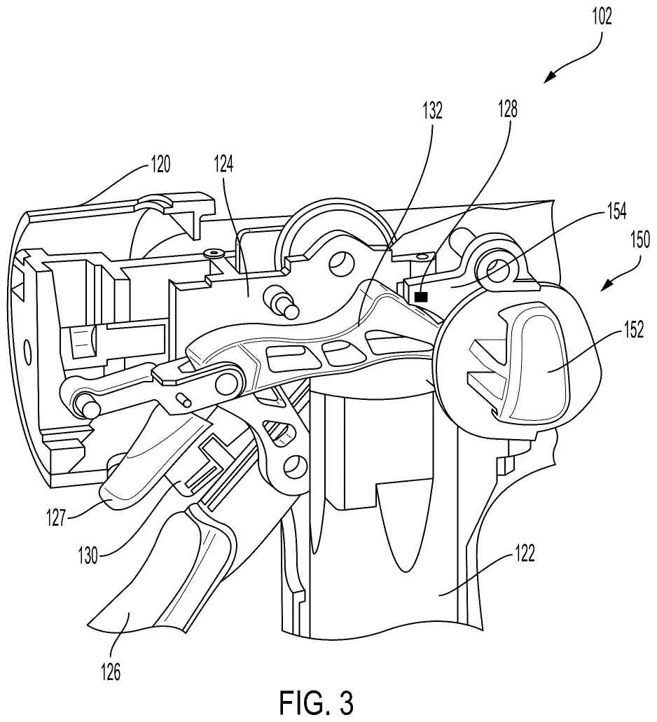

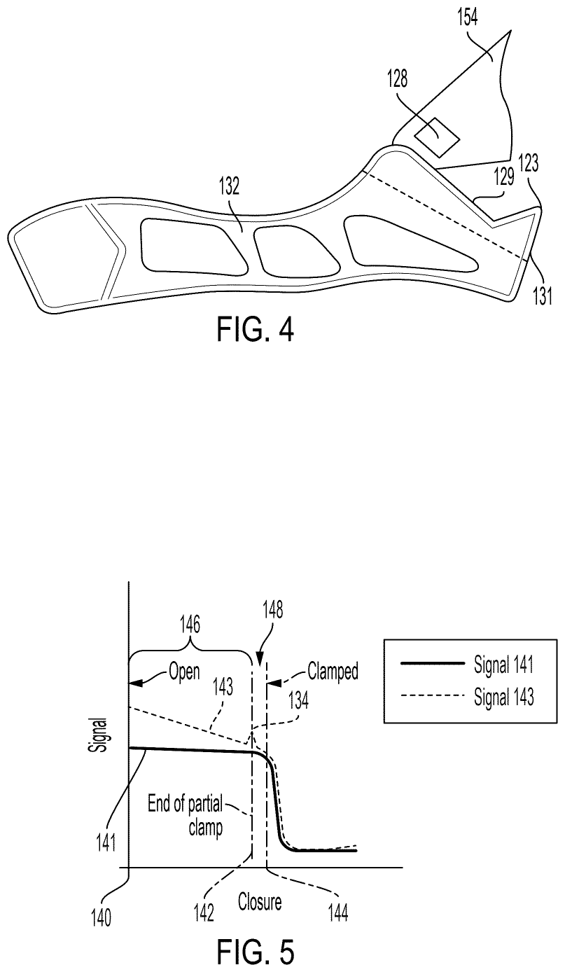

As shown in FIGS. 3 and 4, the frame 124 supports an arm or link 132, which extends proximally toward a closure release assembly 150. In various instances, the clamp arm 126 is referred to as a closure trigger, and the link 132 is referred to as a closure link; the closure link moves with the closure trigger through the closure trigger's closure stroke to move the end effector 108 to the clamped configuration. The link 132 is connected to the clamp arm 126 such that pivotal actuation of the clamp arm 126 toward the pistol grip 122 by a clinician pulls the link 132 relative to the closure release assembly 150.

The link 132 interacts with the closure release assembly 150 to mechanically lock and unlock the clamp arm 126. More specifically, the link 132 protrudes from the clamp arm 126 and includes a locking wall 131 that is configured to cooperate with the closure release assembly 150 pivotally coupled to the frame 124. The closure release assembly 150 includes a release actuator 152 that has a distally-protruding cam follower arm 154 formed thereon. The release actuator 152 can be biased into engagement with the link 132 by a spring, for example. As the clinician depresses the clamp arm 126 to move the clamp arm 126 from its unactuated position towards the pistol grip 122 of the replaceable handle 102, the link 132 pivots upward to a point wherein the cam follower arm 154 of the release actuator 152 drops to meet the locking wall 131 on the link 132 to prevent or block the clamp arm 126 from returning to the unactuated position. Thus, the closure release assembly 150 serves to mechanically lock the clamp arm 126 in the fully-actuated position by blocking the clamp arm's return path from the fully-actuated position.

When the clinician desires to unlock the clamp arm 126 to permit it to be biased to the unactuated position, the clinician simply pivots the release actuator 152 such that the cam follower arm 154 is moved out of engagement with the locking wall 131 on the clamp arm 126. When the cam follower arm 154 has been moved out of engagement with the clamp arm 126, the clamp arm 126 is unlocked or released to pivot back to the unactuated position. Other closure actuator lock and release arrangements may also be employed.

Referring primarily to FIG. 3, the magnet 128 is mounted on the release actuator 152. Actuation of the clamp arm 126 causes the link 132 to move along the release actuator 152 and the profile of the link 132 pushes or cams against the cam follower arm 154 of the release actuator 152 to relocate the magnet 128 relative to the frame 124 and the sensor 118. Referring to the profile of the link 132 in FIG. 4, the link 132 includes a ramp 129 which slopes in a first direction (e.g. downward) to a spike 123 which extends in a second direction (e.g. upward) and defines an abrupt geometry change compared to the slope of the ramp 129. The magnet 128 is mounted or otherwise fixed to the cam follower arm 154, which rides along the ramp 129 (e.g. gradually downward along the ramp 129 and abruptly upward, then downward at the spike 123) until the release actuator 152 pivots to catch the locking wall 131 of the link 132 with the cam follower arm 154.

As further described herein, actuation of the clamp arm 126 causes the magnet 128 to translate. The magnet 128 may move along a linear or arcuate path as the clamp arm 126 is moved through its actuation motion. In various embodiments, rotation of the clamp arm 126 may cause the magnet 128 to relocate in multiple planes, which can increase the distance between the magnet 128 and the sensor 118 during the stroke.

The clamp arm 126 is movable through a plurality of discrete motions or zones during the stroke. The motions correspond to different operational states of the clamp arm 126, which are indicative of corresponding operational states of the surgical instrument 100. For example, the clamp arm 126 is movable through discrete states including an open state, a fully-clamped state, and a partially-clamped state intermediate the open state and the fully-clamped state. Threshold positions of the clamp arm, or closure trigger, correspond to a transition between the open state and the partially-clamped state, and between the partially-clamped state and the fully-clamped state. Additional and/or alternative closure states and threshold transitions are also contemplated.

As one example, the surgical instrument 100 may prevent articulation of the end effector 108 after the tissue has been clamped between the first and second jaws 112, 114. Articulation of the end effector 108 when it is fully clamped onto tissue could pull the tissue clamped by the end effector 108 as the end effector is articulated. For example, an electronic lockout can prevent the articulation motion when the end effector 108 is in a fully-clamped configuration, which can be determined as further described herein, i.e. based on detection of a transition between operational states and into the fully-clamped state, for example.

The position of the clamp arm 126 may be indicative of the closure state of the surgical instrument 100. The closure state of the surgical instrument 100, and of the end effector 108 attached thereto, may be determined by the circuit board 116 based on a signal detected by the sensor 118 of the reusable housing 104. Initially, when the reusable housing 104 is first mounted to the replaceable handle 102, the magnet 128 may be positioned a first distance away from the sensor 118, which corresponds to an open state for the surgical instrument 100, and results in a first magnetic field strength. As the clamp arm 126 moves through the trigger stroke, which causes the magnet 128 to move relative to the frame 124, the strength of the magnetic field may change, which may cause the strength of the signal detected by the sensor 118 to change.

More specifically, the sensor 118 is a magnetic sensor, such as a Hall effect sensor, which is configured to detect a magnetic field generated by the magnet 128. The strength of the signal detectable by the sensor 118 is proportional to the strength of the magnetic field received by the sensor 118. For example, as the distance between the magnet 128 and the sensor 118 decreases, the strength of the magnetic field, and, therefore, the signal, detected by the sensor 118 may increase. Conversely, as the distance between the magnet 128 and the sensor 118 is increased, the strength of the magnetic field and the strength of the signal detected by the sensor 118 may decrease. Because actuation of the clamp arm 126 relocates the magnet 128 relative to the frame 124 (via motion of the link 132) and relative to the sensor 118 in the replaceable handle 102 mountingly fixed relative to the frame 124, the strength of the magnetic field and the signal detected by the sensor 118 is indicative of the location of the magnet 128 and, in turn, the location of the clamp arm 126. The circuit board 116 logic is programmed to determine the position of the clamp arm 126 based on the signal detected by the sensor 118.

In various instances, a clinician can actuate the clamp arm 126, such that one or both of the first and second jaws 112, 114 of the end effector 108 are moved to clamp tissue therebetween. If the clinician wants to reposition the end effector 108, the clinician may release the clamp arm 126 to reopen the first and second jaws 112, 114 of the end effector 108, reposition the end effector 108, and then, re-actuate the clamp arm 126 to re-clamp the end effector 108. The logic of the circuit board 116 can determine the directionality of movement of the clamp arm 126 (e.g., whether the clamp arm 126 is being actuated or released). In various embodiments, the directionality may be based on the strength of the signal and/or a comparison of the strength of the signal with respect to one or more previously detected signals (e.g. increasing or decreasing). In such instances, the directionality of movement of the clamp arm 126 can be determined independent of encoders or sensors monitoring the direction of rotation of the closure and/or firing motors.

Referring now to FIG. 5, exemplary voltage signal curves are depicted. The voltage signal curves depict analog voltage from the sensor 118 (e.g. a Hall Effect sensor) based on movement of the magnet 128 during the closure stroke. A first voltage signal 141 corresponds to an exemplary voltage signal for a closure link with the modified geometric profile indicated with the dashed line in FIG. 4. In such instances, the signal 141 detected by the sensor 118 is constant (absent noise in the system) during a partially-clamped state 146 between a first position 140 of the clamp arm 126, which corresponds to an open configuration of the end effector 108, and a second position 142 of the clamp arm 126, which corresponds to the end of a partially-clamped configuration of the end effector 108. When the clamp arm 126 is fully-actuated at a third position 144, which corresponds to a fully-clamped configuration, the signal 141 drops suddenly in the fully-clamped state 148 based on the magnet suddenly being relocated away from the sensor 118. For example, the magnet can be positioned on the cam follower arm 154, which is positioned and structured to drop downward away from the sensor 118 when the clamp arm 126 is fully clamped to lock the clamp arm 126 in the fully-clamped configuration, as further described herein. The locking engagement between the cam follower arm 154 of the closure release assembly 150 and the locking wall 131 on the link 132 corresponds to a perturbance along the magnet's path, which causes the abrupt change in the first voltage signal 141. A perturbance refers to a sudden change in direction or trajectory of the magnet's path, which causes an abrupt signal change.

Referring still to FIG. 5, a second voltage signal 143 corresponds to an exemplary voltage signal for the magnet 128 (FIGS. 3 and 4) during a closure stroke. In such instances, owing to the geometry of the link 132 and, in particular, to the profile of the ramp 129 and the spike 123 engaged with the release actuator 152, the signal 143 is indicative of the position and directionality of the clamp arm 126. Owing to the ramp 129, the signal 143 gradually decreases during an initial portion of the closure stroke from the first position 140 of the clamp arm 126 to the second position 142 of the clamp arm 126 indicating the magnet's 128 linear path relative to the sensor 118 throughout the partially-clamped state 146. The slope or incline of the ramp 129 can generate the gradual voltage change through the partially-clamped state 146. Thereafter, similar to the first signal 141, the second signal 143 also drastically drops when the clamp arm 126 is fully clamped at the third position 144.

To further discretize the closure states, the surgical instrument 100 can subject the magnet 128 to the spike 123 during the closure stroke indicative of a transition between closure states of the surgical instrument 100. The spike 123 is structured to relocate the magnet 128 relative to the frame 124 (and, thus, the sensor 118) more drastically than the magnet 128 is otherwise moved or relocated, such that the degree of movement of the magnet 128 produces a significant voltage spike 134 detectable by the sensor 118 and determined, by the control circuit of the circuit board 116, to be indicative of a change in operational states. Referring still to FIG. 5, for example, the second signal 143 includes a detectable voltage spike 134 at the second position 142 of the clamp arm 126 corresponding to the transition from the partially-clamped state 146 to the fully-clamped state 148. The spike 123 causes the distance between the magnet 128 and the sensor 118 to suddenly and significantly change, then suddenly return or revert to the previous measurement, resulting in an abrupt spike in the strength of the signal 143. As such, the voltage spike 134 resulting from the magnet 128 be moved relative to the sensor 118 based on the geometry of the spike 123 is indicative of the clamp arm 126 transitioning out of the partially-clamped state 146 and into the fully-clamped state 148. The sudden change in direction/trajectory of the magnet during the closure stroke can be referred to as perturbance in the magnet's path.

By using the spike 123 to generate the voltage spike 134, the analog voltage detected by the sensor 118 includes a measurable step or jump, rather than a continuous, linear progression. Although residual noise may still be present in the signal detected by the sensor 118, the voltage spike 134 may provide a clearer indication of the discrete state of the surgical instrument as compared to a linear signal. The voltage spike 134 is a rapid change in voltage detected by the sensor 118. For example, the voltage spike 134 can occur nearly instantaneously or quickly in time (e.g. fractions of a second). In various instances, the voltage spike 134 can occur in a small portion of the overall closure stroke length (e.g. less than 5%, less than 2%, or less than 1% of the closure stroke).

The reader will appreciate that alternative geometries and signals are contemplated. For example, a sudden voltage dip or drop can also be utilized to indicate a change in operational states. Whether the voltage increases or decreases depends on, for example, the orientation of the positive and negative pole of the magnet 128.

The reader will appreciate that the voltage curves of FIG. 5 are exemplary. Alternative closure link geometries, release actuator geometries, and/or placement of the magnet 128 and/or the sensor 118 can generate different signals that convey the movement and directionality of the closure stroke and/or convey motion through predefined sub-strokes of the firing stroke (e.g. through a range of motion corresponding to a partially-clamped state).

In various aspects of the present disclosure, the reusable housing 104 supports the circuit board 116 and a plurality of sensors, including the sensor 118 and a second sensor 119. In certain instances, the second sensor 119 can also be a magnetic sensor like a Hall Effect sensor, for example. The sensors 118, 119 are in signal communication with a processor and memory of the circuit board 116 to determine a closure state of the surgical instrument 100 based on the signals. For example, the magnet 128 can be moved in multiple dimensions and feedback from both sensors 118, 119 can be monitored to determine the direction of the magnet's movement and, thus, the direction of the stroke (e.g. to open the jaws or to close the jaws). In one aspect of the present disclosure, the sensor 118 can detect the directionality of the stroke, and the second sensor 119 can detect the distance displaced during the stroke, or vice versa.

Referring again to FIGS. 2 and 3, the magnet 128 is mounted to the release actuator 152 and movable with the clamp arm 126, which is actuated to open and close at least one of the first and second jaws 112, 114 of the end effector 108. In other aspects of the present disclosure, the magnet 128 can be mounted on another feature movable with the clamp arm 126, such as the link 132 or another extension or arm connected (directly or indirectly) to the clamp arm 126, for example, to monitor the movement of the clamp arm 126 through the closure stroke. In still other aspects, a magnet can be mounted to another actuator of the replaceable handle 102 to monitor the position of the other actuator with respect to threshold positions and/or operational states. For example, the replaceable handle 102 further includes a firing trigger 127 for the firing drive system, which is actuated to advance a firing driver to eject staples and push a knife for cutting. It will be appreciated that instruments having end effectors directed to different surgical tasks may have different numbers and/or types of actuators or other suitable controls for operating the end effector and the stroke of those actuators can be monitored by the sensor arrangements disclosed herein.

In other aspects of the present disclosure, a voltage signal can include multiple abrupt signal changes, which can be indicative of transitions between operational states. In various instances, a closure actuator or trigger, such as the clamp arm 126, for example, can include a stepped profile that engages and displaces a movable arm during a closure stroke to indicate different operational states and transitions therebetween.

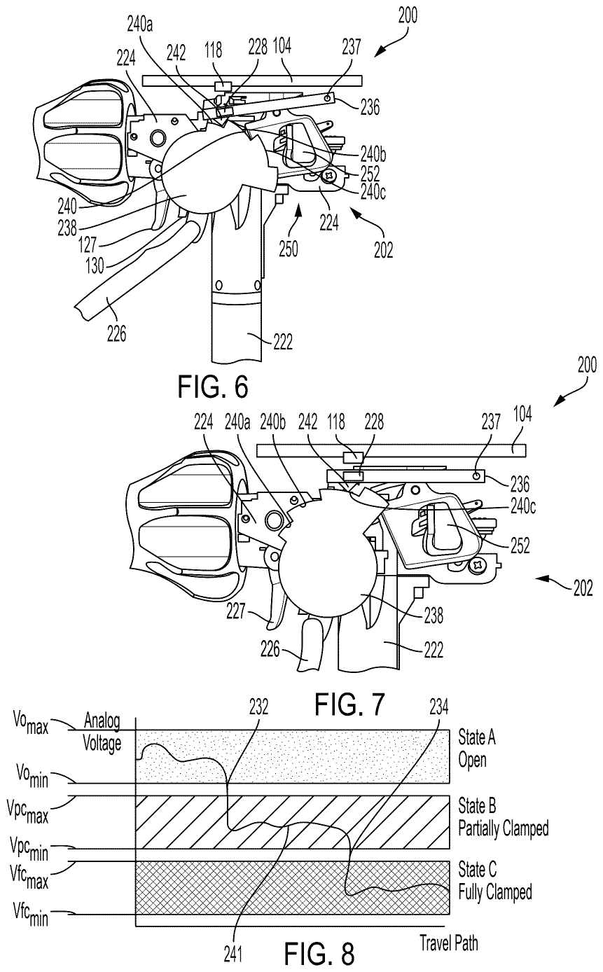

Referring now to FIGS. 6 and 7, a portion of a surgical instrument 200 is shown. The surgical instrument 200 is similar to the surgical instrument 100 in many aspects and includes, for example, the end effector 108 and the shaft 106 (FIG. 1). The surgical instrument 200 further includes a replaceable handle 202, which is similar to the replaceable handle 102 in many aspects and further includes a cam 238 movable with a clamp arm 226, which is pivotable relative to a pistol grip 222 and a frame 224. The cam 238 operably engages a closure release assembly 250 including a release actuator 252 thereof to selectively lock and unlock the clamp arm 226. The replaceable handle 102 further includes an arm 236 supported by the frame 224 and a magnet 228 is mounted to the arm 236.

The clamp arm 226 is connected to the cam 238 such that the cam 238 rotates as the clamp arm 226 pivots between an actuated position and an actuated position to clamp the end effector 108. Rotation of the clamp arm 226 in a first direction, e.g. counterclockwise in FIG. 6 and toward the pistol grip 222, rotates the cam 238 in a first direction. Release of the clamp arm 226 allows the clamp arm 226 to rotate in a second direction opposite the first direction, e.g. clockwise in FIG. 6 and away from the pistol grip 222, which rotates the cam 238 in a second direction.

The cam 238 includes a stepped profile 240 including the steps 240a, 240b, and 240c, which engage the arm 236 during the closure motion. More specifically, the arm 236 is supported by and engaged with the cam 238 such that a portion of the arm 236 is moved by rotation of the cam 238. In various instances, arm 236 is supported at a pivot 237 and includes a tooth 242 that rides along the cam 238. Rotation of the cam 238 can lift (or lower) an end of the arm 236 to pivot the arm 236 relative to the frame 224. In various instances, the arm 236 is biased into engagement with the cam 238 such as by a spring, for example.

As the clamp arm 226 is actuated, the cam 238 rotates and the arm 236 slides along the cam 238. For example, as shown in FIG. 6, when the arm 236 is engaged with the first step 240a of the cam 238, the magnet 228 (supported on the arm 236) and the sensor 118 are separated by a first distance. As shown in FIG. 7, when the arm 236 is engaged with the third step 240c of the cam 238, the magnet 228 and the sensor 118 are separated by a second distance, which is less than the first distance. In such instances, the strength of the magnetic field detected by the sensor 118 is dependent on the changing distance between the magnet 228 and the sensor 118.

The stepped profile 240 of the cam 238 is designed to move the magnet 228 relative to the sensor 118 to create a series of voltage changes at predefined locations along the closure stroke, such as at the transition between operating states, for example. As shown in FIG. 8, the signal 241 detected by the sensor 118 is the analog voltage detected by the sensor 118, which is proportional to the strength of the magnetic field generated by the magnet 228. Similar to the voltage signals 141, 143 (FIG. 5), the signal 241 changes as the clamp arm 226 moves through the closure stroke, which causes the magnet 228 to move relative to the frame 224 and relative to the sensor 118. The stepped profile 240 generates a series of step-wise voltage changes through the closure stroke corresponding to the different closure states. For example, engagement with the first step 240a can correspond to an open state of the surgical instrument 200, engagement with the second step 240b can correspond to the partially-clamped state of the surgical instrument 200, and engagement with the third step 240c can correspond to the fully-clamped state of the surgical instrument 200.

As an example, the clamp arm 226 can initially be in an unactuated position corresponding to the end effector 108 being open. In this initial position, the first step 240a of the cammed profile 240 is cammingly engaged with the arm 236. As the clamp arm 226 is pivoted toward the pistol grip 222 to initiate closure of the end effector 108, the cammed profile 240 of the cam 238 rotates such that the second step 240b moves into engagement with the tooth 242 of the arm 236 to lift the arm 236 from the first step 240a to the second step 240b. Movement of the arm 236 up and across the riser between the steps 240a, 240b causes an abrupt voltage change 232 indicative of the transition from the open state to the partially-clamped state. Similarly, continued actuation of the clamp arm 226 toward the pistol grip 222 causes the cammed profile 240 to continue to rotate relative to the tooth 242 such that the third step 240c moves into engagement with the tooth 242 of the arm 236 to lift the arm 236 from the second step 240b to the third step 240c. Movement of the tooth 242 up and across the riser between the steps 240b, 240c causes another abrupt signal change 234 indicative of the transition from the partially-clamped state to the fully-clamped state.

The voltage changes 232, 234 are detectable, significant voltage changes. For example, the voltage changes 232, 234 are larger than a change in voltage attributable to signal noise. Whether a change in detected voltage constitutes a voltage spike 134 can be dependent on the amount of time in which the voltage is changed. For example, if the change in voltage occurs very quickly (e.g., approximately instantaneously), the change in voltage may be considered a voltage change that is indicative of a change in the closure state. If the change in voltage occurs slowly (e.g., over a period of time), the change in voltage may be indicative of general movement of the clamp arm 226. In FIG. 8, the voltage changes 232, 234 are shown as sudden decreases in the detected voltage. In other aspects of the present disclosure, the voltage changes 232, 234 can be a sudden increase and/or decrease in the detected voltage.

Voltages changes 232, 234 are rapid changes. For example, the voltages changes 232, 234 can occur nearly instantaneously or quickly in time (e.g. fractions of a second). In various instances, the voltage changes 232, 234 can occur in a small portion of the overall closure stroke length (e.g. less than 5%, less than 2%, or less than 1% of the closure stroke). In various instances, the duration and/or degree of a voltage change can be compared to a threshold value stored in the memory of the control circuit of the circuit board 116 to determine if the voltage change amounts to a voltage spike corresponding to a change in operational states of the surgical instrument 200.

In certain instances, the circuit board 116 can be programmed to compare the detected voltage to predetermined voltage ranges stored in a memory to determine the closure state. Referring again to FIG. 8, the circuit board 116 can be programmed to determine that the surgical instrument 100 is in an open state if the detected voltage is between a maximum open voltage voₘₐₓ and a minimum open voltage voₘᵢₙ; the circuit board 116 may determine that the surgical instrument is in a partially clamped state if the detected voltage is between a maximum partially clamped voltage vpcₘₐₓ and a minimum partially clamped voltage vpcₘᵢₙ; the circuit board 116 may determine that the surgical instrument is in a fully clamped state if the detected voltage is between a maximum fully clamped voltage vfcₘₐₓ and a minimum fully clamped voltage vfcₘᵢₙ. In various instances, the minimum open voltage voₘᵢₙ may be greater than the maximum partially-clamped voltage vpcₘₐₓ; the minimum partially-clamped voltage vpcₘₐₓ may be greater than the maximum fully-clamped voltage vfcₘₐₓ. As such, the range of voltage indicative of the fully-open state, voₘₐₓ to voₘᵢₙ, may greater than the range of voltages indicative of the partially-clamped state, vpcₘₐₓ to vpcₘᵢₙ, and the range of voltages indicative of the partially-clamped state, vpcₘₐₓ to vpcₘᵢₙ, may be greater than the range of voltages indicative of the fully-clamped state, vfcₘₐₓ to vfcₘᵢₙ. In other instances, the voltage can increase from the open state to the partially-state and/or from the partially-clamped state to the fully-clamped state.

The present disclosure further contemplates that the relationship between the voltages may be different such as, for example, the range of voltages indicative of the fully clamped state, vfcₘₐₓ to vfcₘᵢₙ, may be larger than the range of voltages indicative of the partially clamped state, vpcₘₐₓ to vpcₘᵢₙ, and the range of voltages indicative of the partially clamped state, vpcₘₐₓ to vpcₘᵢₙ, may be greater than the range of voltages indicative of the open state, voₘₐₓ to voₘᵢₙ. In various instances, the magnet 228 can move farther from the sensor 118 as the clamp arm 226 is actuated to clamp tissue. Whether the voltage increases or decreases during the closure stroke depends on the positioning of the magnet 228 relative to the sensor 118 and the orientation of the magnet's positive or negative poles.

Alternative cam geometries are contemplated. For example, instead of a stepped profile, the cam 238 can comprise a ramped or inclined cam surface, along which the arm 236 rides during at least a portion of the closure stroke. The ramped cam surface can further define at least one protrusion, detent, or ledge to indicate the transition between operating states. The sloped profile can indicate the directionality of the clamp arm's stroke and the protrusion can indicate a change in operational state, for example.

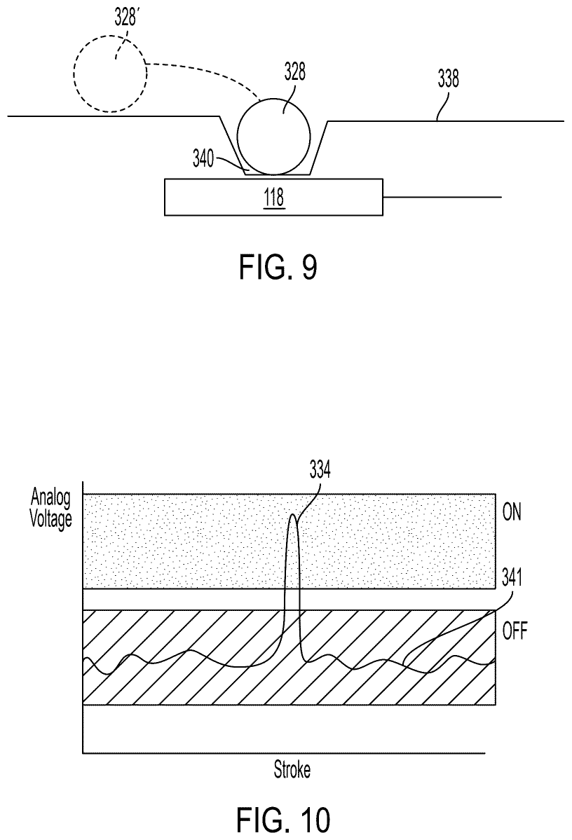

Additionally or alternatively, referring to FIG. 9, a surgical instrument can include a pawl 328, which is flexibly supported relative to the frame of the reusable housing. For example, the pawl 328 can be supported by a spring and/or elastomeric fastener, which flexibly holds the pawl 328 relative to a movable structure 338 (e.g. the clamp arm, closure link, or release actuator movable by the clamp arm) during at least a portion of the stroke. For example, the movable structure 338 can define a detent, or ledge, 340 along a surface thereof, and the pawl 328 can drop into the detent 340 during the stroke to create a sudden shift in the voltage detected by the sensor 118. In various instances, the pawl 328 can be biased into engagement with the movable structure 338 and detent 340 therein. The pawl 328 can be a magnet, which moves relative to the sensor 118 to create a voltage spike during the stroke, as further described herein. The pawl 328 can comprise a spherical or non-spherical geometry.

As shown in FIG. 10, the interaction of the pawl 328 and the detent 340 produces a voltage spike 334 in the signal 341 during the closures stroke. The spike 334 corresponds to the transition between closure states.

In other instances, a piezoelectric element can be used instead of a magnetic sensor. For example, features of the surgical instrument can interact during at least a portion of the closure stroke, and the mechanical pressure from the interaction can be converted to electricity spikes denoting the different operational states and threshold transitions therebetween. More specifically, referring again to FIG. 9, the pawl 328 and the detent 340 can form a piezoelectric sensor to monitor the closure stroke based on the interaction between the pawl 328 and the detent 340. In such instances, the sensor 118 can detect an electrical impulse to characterize a particular threshold of the closure stroke, such as the transition from partially-closed to fully-closed.

The figures and description herein describe, for example, a magnetic sensor for detecting a closure state of the end effector (e.g. open, partially-clamped, fully-clamped end effector jaws); however, the reader will appreciate that the magnetic sensors herein can be utilized to monitor additional operational states and parameters of the end effector, such as the firing stroke and/or articulation motion, for example. Moreover, the magnetic sensors herein can be utilized in different surgical devices, such as graspers, cutters, clip appliers, access devices, drug/gene therapy delivery devices, and/or electrosurgical devices, such as ultrasound, RF, and/or laser energy devices, for example, to monitor various surgical functions thereof.

Examples of the devices, systems, and methods disclosed herein, according to various aspects of the present disclosure, are provided below in the following numbered clauses. An aspect of the devices, systems, and methods may include any one or more than one, and any combination of, the numbered clauses described below.

Clause 1. A surgical instrument, comprising a handle comprising a frame supporting a trigger and a magnet, wherein an actuation of the trigger relocates the magnet relative to the frame; and a reusable housing supporting a circuit and a sensor in signal communication with the circuit, wherein the reusable housing is detachably mounted to the handle and the sensor is to detect a signal based on relocation of the magnet, and wherein the circuit is to determine directionality and degree of movement of the trigger based on the signal detected by the sensor.

Clause 2. The surgical instrument of Clause 1, wherein the surgical instrument is a surgical stapler and the trigger is a clamp arm.

Clause 3. The surgical instrument of Clause 2, wherein the circuit is to further determine a closure state of the surgical stapler based on the signal detected by the sensor.

Clause 4. The surgical instrument of Clause 2, wherein the surgical stapler further comprises an end effector comprising a first jaw and a second jaw, wherein the actuation of the clamp arm is configured to move at least one of the first jaw and the second jaw to clamp tissue therebetween.

Clause 5. The surgical instrument of Clause 1, wherein the circuit is to further determine a closure state of the surgical instrument based on the signal detected by the sensor, wherein the closure state comprises a partially clamped state intermediate an open state and a fully clamped state.

Clause 6. The surgical instrument of Clause 1, wherein the signal comprises a voltage change as the trigger moves through a trigger stroke.

Clause 7. The surgical instrument of Clause 6, wherein the signal comprises a voltage spike upon the magnet being subjected to a perturbance upon relocation of the magnet relative to the frame, wherein the perturbance is indicative of a change in a closure state of the surgical instrument.

Clause 8. The surgical instrument of Clause 7, wherein the frame of the handle further comprises an arm supporting the magnet, wherein the trigger comprises a cam comprising a step, and wherein the arm slidably engages the step of the cam to generate the perturbance along a path of the magnet.

Clause 9. The surgical instrument of Clause 7, further comprising a closure link movable with the trigger, wherein the closure link comprising a ramp and a spike; the surgical instrument further comprising a closure release actuator supporting the magnet, wherein the closure release actuator slidably engages the ramp and the spike during at least a portion of a trigger stroke, and wherein the perturbance is generated by the closure release actuator sliding across the spike.

Clause 10. The surgical instrument of Clause 1, where the sensor comprises a Hall effect sensor.

Clause 11. A surgical instrument, comprising: a handle comprising a frame supporting a trigger and a magnet, wherein an actuation of the trigger relocates the magnet relative to the frame; and a reusable housing detachably mounted to the handle, the reusable housing supporting a circuit, a first sensor to detect a first signal based on a positional movement of the magnet, and a second sensor to detect a second signal based on a directional movement of the trigger; wherein the first sensor and the second sensor are in signal communication with the circuit, and wherein the circuit is to determine a closure state of the surgical instrument based on the first signal and the second signal.

Clause 12. The surgical instrument of Clause 11, wherein the closure state comprises a partially clamped state intermediate an open state and a fully clamped state.

Clause 13. The surgical instrument of Clause 11, wherein the first signal comprises a voltage change as the trigger moves through a trigger stroke.

Clause 14. The surgical instrument of Clause 13, wherein the first signal comprises a voltage spike upon the magnet being subjected to a perturbance, wherein the perturbance is indicative of a change in the closure state.

Clause 15. The surgical instrument of Clause 14, further comprises an arm supporting the magnet, wherein the trigger further comprises a cam comprising a step, and wherein the perturbance is generated by the arm traversing the step of the cam.

Clause 16. The surgical instrument of Clause 14, further comprising a closure link movable with the trigger, wherein the closure link comprising a ramp and a spike; the surgical instrument further comprising a closure release actuator supporting the magnet, wherein the closure release actuator slidably engages the ramp and the spike during at least a portion of a trigger stroke, and wherein the perturbance is generated by the closure release actuator sliding across the spike.

Clause 17. A surgical instrument, comprising: a handle comprising a frame supporting a trigger and a magnet, wherein an actuation of the trigger relocates the magnet relative to the frame; a reusable housing supporting a circuit, wherein the reusable housing is detachably mounted to the handle; and a sensor in signal communication with the circuit, wherein the sensor is to detect a signal based on movement of the magnet; wherein the circuit is to determine directionality and degree of movement of the trigger based on the signal detected by the sensor.

Clause 18. The surgical instrument of Clause 17, wherein the circuit is to further determine a closure state of the surgical instrument based on the signal detected by the sensor, wherein the closure state comprises a partially clamped state intermediate an open state and a fully clamped state.

Clause 19. The surgical instrument of Clause 17, wherein the signal comprises a voltage change as the trigger moves through a trigger stroke.

Clause 20. The surgical instrument of Clause 19, wherein the signal comprises a voltage spike upon the magnet being subjected to a perturbance upon relocation of the magnet relative to the frame, wherein the perturbance is indicative of a change in a closure state of the surgical instrument.

It should be appreciated that spatial terms such as vertical, horizontal, right, left etc., are given herein with reference to the figures assuming that the longitudinal axis of the surgical instrument is co-axial to the central axis of the shaft, with the triggers extending downwardly at an acute angle from the bottom of the handle. In actual practice, however, the surgical instrument may be oriented at various angles and as such these spatial terms are used relative to the surgical instrument itself. Further, proximal is used to denote a perspective of a clinician who is behind the handle who places the end effector distal, or away from him or herself. As used herein, the phrase, “substantially transverse to the longitudinal axis” where the “longitudinal axis” is the axis of the shaft, refers to a direction that is nearly perpendicular to the longitudinal axis. It will be appreciated, however, that directions that deviate some from perpendicular to the longitudinal axis are also substantially transverse to the longitudinal axis.

Unless specifically stated otherwise as apparent from the foregoing disclosure, it is appreciated that, throughout the present disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-20. (canceled)

21. A surgical instrument, comprising:
a handle comprising a frame supporting a trigger and a magnet, wherein an actuation of the trigger relocates the magnet relative to the frame; and
a reusable housing supporting a circuit and a sensor in signal communication with the circuit, wherein the reusable housing is detachably mounted to the handle and the sensor is to detect a signal based on relocation of the magnet, and wherein the circuit is to determine directionality and degree of movement of the trigger based on the signal detected by the sensor.

22. The surgical instrument of claim 21, wherein the surgical instrument is a surgical stapler and the trigger is a clamp arm.

23. The surgical instrument of claim 22, wherein the circuit is to further determine a closure state of the surgical stapler based on the signal detected by the sensor.

24. The surgical instrument of claim 22, wherein the surgical stapler further comprises an end effector comprising a first jaw and a second jaw, and wherein the actuation of the clamp arm is configured to move at least one of the first jaw and the second jaw to clamp tissue therebetween.

25. The surgical instrument of claim 21, wherein the circuit is to further determine a closure state of the surgical instrument based on the signal detected by the sensor, and wherein the closure state comprises a partially clamped state intermediate an open state and a fully clamped state.

26. The surgical instrument of claim 21, wherein the signal comprises a voltage change as the trigger moves through a trigger stroke.

27. The surgical instrument of claim 26, wherein the signal comprises a voltage spike upon the magnet being subjected to a perturbance upon relocation of the magnet relative to the frame, and wherein the perturbance is indicative of a change in a closure state of the surgical instrument.

28. The surgical instrument of claim 27, wherein the frame of the handle further comprises an arm supporting the magnet, wherein the trigger comprises a cam comprising a step, and wherein the arm slidably engages the step of the cam to generate the perturbance along a path of the magnet.

29. The surgical instrument of claim 27, further comprising a closure link movable with the trigger, wherein the closure link comprising a ramp and a spike; the surgical instrument further comprising a closure release actuator supporting the magnet, wherein the closure release actuator slidably engages the ramp and the spike during at least a portion of a trigger stroke, and wherein the perturbance is generated by the closure release actuator sliding across the spike.

30. The surgical instrument of claim 21, where the sensor comprises a Hall effect sensor.

31. A surgical instrument, comprising:
a handle comprising a frame supporting a trigger and a magnet, wherein an actuation of the trigger relocates the magnet relative to the frame; and
a reusable housing detachably mounted to the handle, the reusable housing supporting a circuit, a first sensor to detect a first signal based on a positional movement of the magnet, and a second sensor to detect a second signal based on a directional movement of the trigger;
wherein the first sensor and the second sensor are in signal communication with the circuit, and wherein the circuit is to determine a closure state of the surgical instrument based on the first signal and the second signal.

32. The surgical instrument of claim 31, wherein the closure state comprises a partially clamped state intermediate an open state and a fully clamped state.

33. The surgical instrument of claim 31, wherein the first signal comprises a voltage change as the trigger moves through a trigger stroke.

34. The surgical instrument of claim 33, wherein the first signal comprises a voltage spike upon the magnet being subjected to a perturbance, and wherein the perturbance is indicative of a change in the closure state.

35. The surgical instrument of claim 34, further comprises an arm supporting the magnet, wherein the trigger further comprises a cam comprising a step, and wherein the perturbance is generated by the arm traversing the step of the cam.

36. The surgical instrument of claim 34, further comprising a closure link movable with the trigger, wherein the closure link comprising a ramp and a spike; the surgical instrument further comprising a closure release actuator supporting the magnet, wherein the closure release actuator slidably engages the ramp and the spike during at least a portion of a trigger stroke, and wherein the perturbance is generated by the closure release actuator sliding across the spike.

37. A surgical instrument, comprising:
a handle comprising a frame supporting a trigger and a magnet, wherein an actuation of the trigger relocates the magnet relative to the frame;
a reusable housing supporting a circuit, wherein the reusable housing is detachably mounted to the handle; and
a sensor in signal communication with the circuit, wherein the sensor is to detect a signal based on movement of the magnet;
wherein the circuit is to determine directionality and degree of movement of the trigger based on the signal detected by the sensor.

38. The surgical instrument of claim 37, wherein the circuit is to further determine a closure state of the surgical instrument based on the signal detected by the sensor, and wherein the closure state comprises a partially clamped state intermediate an open state and a fully clamped state.

39. The surgical instrument of claim 37, wherein the signal comprises a voltage change as the trigger moves through a trigger stroke.

40. The surgical instrument of claim 39, wherein the signal comprises a voltage spike upon the magnet being subjected to a perturbance upon relocation of the magnet relative to the frame, and wherein the perturbance is indicative of a change in a closure state of the surgical instrument.
